(19) United States
(12) Patent Application Publication
(10) Pub. No.: US 2012/0295835 A1
(43) Pub. Date: Nov. 22, 2012

(54) SGEF CONTROLS MACULAR, CORPUS CALLOSUM AND HIPPOCAMPAL FUNCTION AND DEVELOPMENT, LIVER HOMEOSTASIS, FUNCTIONS OF THE IMMUNE SYSTEM, FEVER RESPONSE ATHEROSCLEROSIS AND TUMOROGENIC CELL GROWTH

(76) Inventor: PIERRE BITOUN, PARIS (FR)

(21) Appl. No.: 13/112,788

(22) Filed: May 20, 2011

(51) Int. Cl. A61K 38/17; A61P 27/02; A61P 25/28; A61P 9/10; A01N 1/02; A61P 37/06; A61P 27/06; A61P 37/04; A61P 1/16; C40B 30/04; A61P 35/00; A61P 29/00; A61P 25/00; A61K 31/7088

(52) U.S. Cl. 514/1.9; 514/1.1; 514/17.7; 514/21.2; 514/20.8; 514/17.8; 435/1.1; 514/44 R; 506/9; 514/19.3; 514/19.4; 514/19.5

(57) ABSTRACT

The invention provides a composition comprising SGEF protein or gene as a therapeutic means to clinical or subclinical defects associated with anomalies of at least one from among the macula, corpus callosum, hippocampus, liver or immune system or feverless response to infection. Methods of diagnosis of such disease and development anomalies are based on detection of mutations of the SGEF gene. The SGEF protein is also used as a preventive or curative treatment of atherosclerosis by local or systemic delivery. The invention also provides a composition comprising an inhibitor of the SGEF gene expression or SGEF protein concentration, as a therapeutic means for glaucoma, osteoarthritis, auto-inflammatory diseases, tumors or cancers.

### BACKGROUND OF THE INVENTION

1. Field of the Invention

This invention relates to the field of genetics. It identifies a gene SGEF that controls the development and function of the retinal macula, the corpus callosum, the hippocampus, the liver, the immune system and inflammation, and is a factor in fever response to infections. Null allele mutations in the gene lead to abnormal development and dysfunction, at clinical or sub-clinical levels. Over-expression of the gene occurs in inflammatory processes, cancer or tumor cells.

2. Description of the Background

Macular retinal dystrophy is a major cause of visual handicap and blindness in children and adults. Several dominant and recessive genetic causes of macular dystrophy have been identified: in vitelliform macular dystrophy or Best disease VMD2 on 11q13 (1), encoding the bestrophin, a chloride channel localized at the basolateral plasma membrane of Retinal Pigment Epithelium (RPE) cells; autosomal recessive Stargardt disease ABCA4 on 1p21-p13, an ATP binding-cassette transporter whose dysfunction poisons the RPE by accumulation of lipofuscin fluorophores; dominant stargardt-like macular dystrophy ELOVL4 on 6q14, encoding a very long chain fatty acid elongase whose dysfunction also causes lipofuscin accumulation in the RPE; North Carolina macular dystrophy localized at 6q14-q16.2, with a variable dominant phenotype with macular drusen and age-related macular degeneration and Bietti's disease with macular crystalline deposits; some rare cases of Stargardt like disease have been linked to mutations in the CNGB3 gene achromatopsia. Nishiguchi, K. M. et al, Hum. Mutat. 25:248-258 (2005).

Occult macular dystrophy, a progressive visual disorder, was recently found to be linked to RP1-like 1 gene. Akahori, M., et al, Am. J. Hum. Genet. 87:424-429 (2010).

Formation of the human macula is poorly understood. Genes involved in macular development have recently been identified by CGH array. Kozulin, P. et al, Mol Vis 15:45-59 (2009).

Corpus callosum agenesis (CCA) is the most common brain anomaly with a reported incidence of 0.7 to 1 per 1000 live births. CCA has been associated with several gene defects: mutations in L1CAM causing HSAS/MASA syndrome with Hydrocephalus, mental retardation, and adducted thumbs syndrome; in KCC3 causing Andermann syndrome with progressive neuropathy and dementia; in ARX causing XLAG causing lissencephaly and intractable epilepsy; in MRPS16 causing fatal lactic acidosis with complex I and IV deficiency and brain malformation (Catala M., Neurochirurgie 49(4):441-448 (2003); in ZFHX1B causing Mowat-Wilson syndrome with Hirschsprung disease; in LRP2 gene causing Donnai-Barrow syndrome with omphalocele, high grade myopia, deafness and nephritis; in WDR2, where gene dysfunction has recently been associated with CCA as well as brain malformations. CCA has also been described as associated with Acrocallosal, Aicardi, Chudley-McCullough, FG, Genito-patellar, Temtamy, Toriello-Carey and Vici syndrome. CCA is occasionally associated with more than 20 other syndromes. About half of these syndromes involve ocular malformations. Paul LK et al., Nat Rev Neurosci. 8(4):287-99 (2007).

O'Driscoll recently identified two individuals in one family having 3q25 deletions associated with CCA. O'Driscoll, M. C. et al, Am J Med Genet A. 152A(9):2145-59 (2010).

The hippocampus and related structures of the medial temporal lobe have a critical role in encoding long-term memory and are also necessary for the maintenance of working memory for novel items and associations. Ranganath, C. and D'Esposito M., Neuron 31(5):865-73 (2001).

Hippocampal hypoplasia has been associated with PROM1, which not only involves macular dystrophy and hippocampus hypoplasia but also cell transformation. Arrigoni FI, et al. Eur J Hum Genet. 19(2):131-7 (2011); Zhu L, et al., Nature. 457(7229):603-7 (2009). Hippocampus hypoplasia and microphthalmia has been associated with SOX2 mutations. Sisodiya S. M. et al, Epilepsia 47:534-542 (2006).

Rho proteins are low-molecular-weight GTP-binding proteins, thus controlling the cycle between GDP and GTP bound states. Binding of GTP “activates” Rho GTPases by inducing structural shifts that support association of effector molecules that transmit downstream signals. RhoG is an ubiquitously expressed GTPase, which shares significant homology with Rac and binds to a number of the same effector proteins. Gauthier-Rouviere, C. et al, Mol. Biol. Cell 9:1379-1394 (1998) and Wennerberg K. et al., Biol. Chem. 277:47810-47817 (2002).

SGEF (SH3-containing Guanine Nucleotide Exchange Factor) is a RhoG guanine nucleotide exchange factor that stimulates macropinocytosis (engulfing of extracellular fluid). Ellerbroek, S M. et al, Mol. Biol. Cell, 15:3309-3319 (2004). Macropinocytosis occurs constitutively in dendritic neural cells for immune surveillance and can be transiently activated in other cells by growth factors. This actin-based process accompanies ruffling of membranes leading to formation of macropinocytic vesicles that engulf large volumes of fluid. This process can be triggered by bacteria (like Salmonella T.) to invade cells. Pollard, T. D. and Earnshaw W. C., Cell Biology, Elsevier Science, Saunders Ed. p. 363 (2004).

The Rho GTPase Switch.

Rho GTPases are targeted to the membrane by posttranslational attachment of prenyl groups by geranyl-geranyltransferases (GGTases). Cycling between the inactive (GDP-bound) and active (GTP-bound) forms is regulated by guanine nucleotide exchange factors (GEFs) and GTPase-activating proteins (GAPs). Guanine-nucleotide dissociation inhibitors (GDIs) inhibit nucleotide dissociation and control cycling of Rho GTPases between membrane and cytosol. Active, GTP-bound GTPases interact with effector molecules to mediate various cellular responses. Upstream activation of the GTPase switch occurs through activation of GEFs. Schmidt A. and Hall, A. Genes Dev.16:1587-1609 (2002).

It would be desirable to find a single gene/gene product which influences the multiple functions and development of the multiple organs described above. Clearly, that would allow early diagnosis and potential treatment of development or functional problems, as well as diagnosis and potential treatment of development or functional problems that are at the subclinical level.

### SUMMARY OF THE INVENTION

In one aspect of the invention, the invention provides a composition comprising at least one isolated or purified SGEF gene in a functional form introduced into a mammal for expression of at least one SGEF protein and a pharmaceutical carrier. The mammal preferably is a human. More preferably, the human has a clinical or subclinical condition for at least one disease from among a disease associated with structure or function of macula, corpus callosum, hippocampus, liver, immune function or feverless infection. In another embodiment, the mammal, has a genetic defect causing reduced or null expression or activity of a natural SGEF protein. In one embodiment, the genetic defect causes reduced or null expression or activity of the SGEF gene located at 3q25.2 of the human genome.

In another aspect, the invention provides a composition comprising at least one isolated or purified SGEF protein variant in a functional form. In one embodiment, the variant is a variant of a SGEF protein encoded by a SGEF gene located at 3q25.2. In a preferred embodiment, the variant is at least one SGEF protein variant is selected from among the protein variants of SEQ ID No 1, SEQ ID No 2, SEQ ID No 3, SEQ ID No 4, or SEQ ID No 5. More preferably, the at least one SGEF protein variant is selected from among the protein variants of SEQ ID No 1, SEQ ID No 2, or SEQ ID No 3.

In yet another aspect, the invention provides a method of treatment comprising providing at least one SGEF protein variant and a pharmaceutical carrier to an individual manifesting a clinical or subclinical condition or predisposition for a disease associated with functional or structural defects corresponding to retina/macula anomaly (“RMA”), corpus callosum anomaly, hippocampus anomaly, liver disease, immune response deficiency or feverless infection. Preferably, the SGEF protein is a SGEF protein corresponding to the protein encoded by the SGEF gene located at 3q25.2. In accordance to one embodiment, the disease state is associated with RMA and comprises at least one disorder from among retinal disorders, macular disorders, macular dystrophies or macular degenerations like age-related macular degeneration, geographic atrophy, diabetic retinopathy, glaucomatous retinal dysfunction and visual disorders. In accordance to another embodiment, the disease state is associated with corpus callosum anomaly and comprises at least one disorder from among hypoplasia, absence or thickened corpus callosum and coordination disorders, including hand eye coordination disorders. In yet another embodiment, the disease state is associated with hippocampal development deficiency or dysfunction and comprises at least one disorder from among memory dysfunction, intellectual deficiency, mental retardation, Alzheimer disease or degenerative brain disorders. In a further embodiment, the disease state is associated with immune response and comprises at least one disorder from among innate or acquired immune deficiency disorder caused by HIV infection, congenital immune deficiencies, ADA (adenosine deaminase), or steroid induced immune deficiency. In a further yet embodiment, the disease state is associated with liver disease and comprises at least one disease from among hepatitis, congenital liver disease, liver cirrhosis or lack of liver homeostasis.

In yet another aspect, the invention provides a method of diagnosis of at least one disease state selected from among retinal macular anomaly (RMA), corpus callosum anomaly, hippocampus anomaly, liver disease, immune dysfunction, or feverless response to infection, comprising identifying a defect in an SGEF gene located at 3q25.2 or reduction in the concentration of an SGEF protein. Preferably, the identification is by hybridization to a probe specific for the SGEF gene, PCR analysis and western blot analysis. In accordance to one embodiment, the diagnosis of an individual comprises the detection of a defect in the SGEF gene located at 3q25.2 in a consanguineous other-individual or manifesting clinical or physical anomaly corresponding to at least one disease state from among retinal macular anomaly (RMA), corpus callosum anomaly (CCA) liver disease, immune dysfunction and feverless response to an infection.

In a yet still another aspect, the invention provides a method of treatment or prevention of atherosclerosis or arteritis of all arteries and more specifically coronary artery disease comprising the modulation of SGEF expression or activity by genetic or pharmacologic means in a mammal.

In a further aspect, the invention provides a method of treatment or prevention of cancer or tumor growth, comprising administration of an agent to reduce SGEF presence or activity in a mammal. In one embodiment, the invention provides a method for prevention of cancer or tumor growth, wherein the cancer or tumor is a prostrate, brain, breast, ovary, oesophageal, gastrointestinal, liver or yet other cancer or tumor.

In still another aspect, a method of treatment is provided, wherein an SGEF inhibitor is provided to a subject. In one embodiment, the invention provides a method of treatment or prevention of osteoarthritis and joint inflammatory processes, comprising administration of an agent to reduce SGEF presence in a mammal, the agent being administered locally or systemically.

In another aspect the invention provides a method of treatment or prevention of inflammatory or auto-inflammatory diseases, illnesses or processes, comprising administration of an agent to reduce SGEF presence or activity in a mammal, the agent being administered locally or systemically.

In a further aspect, the invention provides a method of treatment or prevention of increased intraocular pressure or glaucoma comprising administration of an agent to reduce SGEF presence in a mammal the agent being administered locally and or systemically.

In a yet further still aspect, the invention provides a method of preservation or preparation of an organ for transplantation, wherein said organ is exposed to a solution comprising SGEF protein or protein variant. In accordance to one embodiment, the organ is liver.

In a still yet further aspect, the invention provides a kit for treatment of a patient comprising at least a functional domain of an SGEF protein and a pharmaceutical excipient. In accordance to one embodiment, the protein is provided as a gene for expression in a mammal.

In a yet still further aspect, the invention provides a kit for treatment of a patient comprising at least an inhibitor of an SGEF protein functional domain and a pharmaceutical excipient.

### DETAILED DESCRIPTION

### Drawings

FIG. 1 depicts a genetic analysis identifying the locus of the deletion mutation. The upper panel presents a Comparative Genome Hybridization (CGH) analysis. The probes are indicated as dots. This FIGURE shows the line shift on chromosome 3q25.2 indicating the homozygously decreased genetic material indicated by the missing hybridized probes in this region.

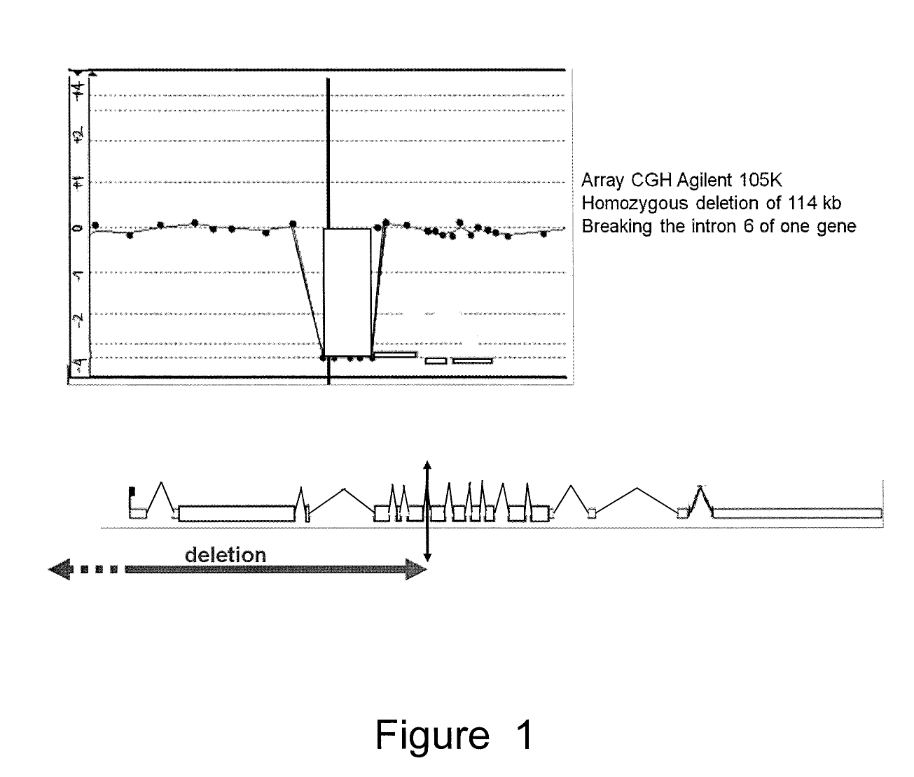

The lower panel of FIG. 1 is a depiction of the SGEF genetic locus. The single line illustrates introns. The boxes represent exons. The black arrow at bottom shows the missing upstream region and first six exons which are removed by the deletion.

### DESCRIPTION

A homozygous null allele SGEF gene mutation has been identified. Unexpectedly, the mutation has revealed the role of SGEF in development of organs and control of multiple functions. The invention provides therapeutic and diagnostic options based on the SGEF gene and protein. The gene mutation reveals the SGEF gene and its product to affect structures and/or functions shown to be associated with retinal macular development; corpus callosum development; hippocampal development; liver function, immune function, and lack of fever response. In contrast, the excess of expression of the gene or increased protein level increases cell multiplication and cell transformation, to play a role in tumor or cancerous growths and atherosclerosis.

This is an unexpected development, because patients simultaneously impaired in these functions are rarely observed. Furthermore, cases of one genetic locus affecting these multiple structure and functions are not known. Without limiting the invention to any theory as to a mechanism of action, it is noted that SGEF is an activator of RhoG, a GTPase protein. There are many known GTPases and the absence of one GTPase regulator might have been considered insufficient to cause multiple syndromes, because it might have been expected that there are separate control mechanisms for GTPase associates with particular functions and/or tissues and, furthermore, it would have been expected that there is redundancy in the control of individual classes of GTPases. There are more than 60 known human Rho GEFs (out of 85 GEF's in the human genome) and each particular GEF determines in which membrane the GTPase is activated and, by acting as a scaffold, which downstream protein the GTPase activates. Alberts B. et al, Molecular Biology of the Cell, Garland Science, N.Y., 5th edition, 927, 931, 1043 (2008).

Nonetheless, a mutant was now shown to have multiple effects. A combination of genetic analysis and clinical and physical observations confirmed the role of the gene. The gene defect was shown to be responsible for defects in both homozygous and heterozygous individuals, establishing its overall role in control of development and function of multiple systems. This points to a dosage sensitivity of SGEF in different parts of the cell at different times and in different tissues to allow proper development of the retinal macula, the corpus callosum, the hippocampus, the liver and immune function, and normal fever response to infection.

Relying on array Comparative Genetic Hybridization (“CGH”) analysis (e.g. as available from Agilent Technologies), the inventor determined that consanguineous parents (cousins) are each heterozygous for a deletion mutation in the SGEF gene located at 3q25.2. The results were confirmed by quantitative PCR analysis. The parents produced three children and all the children are homozygous for the deletion mutation at the 3q25.2 locus. The inventor determined the deletion to cover the same region in all the family members. It is an about 118 kilobase extensive deletion, comprising the 5′-end of the gene including the promoter region and extending into the 6^{th} intron of the SGEF gene and thus abolishing the gene function linked to this major promoter.

The oldest child of this family is Child 1, the middle child and the proband for the genetic study is Child 2. No gross genetic defects were observed upon peripheral lymphocyte karyotypic analysis of the family members.

The proband (Child 2) was initially analyzed for genetic defects because of severe retinal dystrophy, resembling a severe and congenital form of Stargardt's disease. Accordingly, this child and the family members also were analyzed for genetic defects at the ABCA4 (previously called ABCR) locus, a locus known to be associated with Stargart's disease. Allikmets R., Nat Genet 17(1):122 (1997).

To help rule out the coincidence of the mutation at 3q25.2 existing in the background of known mutations associated with macular dysfunction or abnormalities, the proband was tested also for mutations in 18 other known autosomal recessive Retinitis Pigmentosa genes: CERKL, CNGA1, CNGB1, MERTK, PDE6A, PDE6B, PNR, RDH12, RGR, RLBP1, SAG, TULP1, CRB, RPE65, USH2A, USH3A, LRAT, and PROML1. The proband was shown to have two variant isoforms in the ABCA4 locus on the same chromosome (IVS45+7G>A and S2255I) paternally inherited, but no mutations in the other 18 loci. Accordingly, the other four family members were tested for the ABC4 locus mutation. Only the father had the two ABCA4 variants, which he transmitted to the proband. While the S2255I variant is likely a polymorphism, the role of the splice site variant is debated. Valverde, D. et al. Invest Ophthalmol Vis Sci. 48(3):985-90 (2007).

Clinical observation and/or testing revealed the following phenotypes and morphologies (the youngest child was, generally, not tested). The proband had congenital nystagmus and vision impairment with congenital macular dystrophy shown upon fundus examination. Optical Coherence Tomography imaging showed reduced thickness of the retinal macula (92 μm, i.e. about 50% of normal). Further evidence of macular dysfunction and dystrophy were documented by Visual Evoked Potential (VEP) analysis which record visual occipital cortex activity (using occipital cranial electrodes) elicited by light stimulation of each eye.

Prenatal ultrasound had also demonstrated Corpus Callosum (“CC”) agenesis. Magnetic Resonance Imaging demonstrated the complete absence of axonal corpus callosum fibers (white matter), and diminished volume of hippocampus gray matter (hypoplasia) as well as external hydrocephalus (excessive fluid volume outside the brain) were observed in the proband. The proband demonstrated reading and learning difficulties, conditions expected in view of these physical defects.

The proband also had protracted EBV infection lasting for months and the associated mononucleosis, causing severe liver damage (hepatic cytolysis), as well as simultaneous Group A beta-hemolytic streptococcus infection without ever showing signs of fever. The observation regarding the infection and the lack of fever conceptually fits the known function of SGEF in dorsal ruffles formation, i.e. suggesting a trans-endothelial migration role that is involved in the immune response. Accordingly, the immune response is affected by the 3q25.2 locus mutation (SGEF gene). The proband has evidenced the lack of ability to mount a fever response to multiple serious infections including at least a protracted, three-months course of infectious mononucleosis complicated by liver involvement, Group A beta streptococcal infection, tooth abscess, upper respiratory infection etc. This lack of fever is clearly linked to the immune role of SGEF.

Because of the high level of brain expression of SGEF and its role in the cortical and white matter it is likely that SGEF dysfunction mediates the lack of fever which would normally be an aspect of the multiple serious infections observed in the proband. Without limiting the invention to any particular mechanism of action, it is noted that recurrent fevers have been associated with several disorders involving inflammatory conditions, including Familial Mediterranean fever linked to the MEFV gene. Cell 90:797-807 (1997); Houten, S. M. et al. Nature Genet. 22:175-177, (1999). Dominant periodic fever has been associated with the Tumor Necrosis Factor Receptor Super Family 1A, TNFRSF1A. McDermott, M. F. et al, Cell 97:133-144 (1999). A spectrum of auto-inflammatory conditions, the cryopyrinopathies, have been linked to mutations in Cryopyrin, the protein encoded by CIAS1, which activates Caspase 1, which in turn causes release of the active proinflammatory cytokine interleukin-1beta. IL-1beta Ryan J. G. and Kastner, D. L., Curr Top Microbiol Immunol. 321:169-84 (2008).

Lack of fever has been previously observed in familial dysautonomia also known as Riley-Day syndrome which is, like SGEF, involved with cytoskeletal regulation. Cheishvili D. et al., Hum Mol Genet. 2011 Feb. 11. [Epub ahead of print]

Basal body temperature has been linked to serotoninergic receptors 5-HT (1A). Olivier J. D. et al, Eur J Pharmacol 20:590(1-3):190-7 (2008). Basal body temperature is mediated by an 5-HT(1A) receptor population. Bacterial and viral infections induce Hypothalamic Pituitary Axis activation, and also increase brain Nor Epinephrine and 5-HT metabolism and brain tryptophan. These effects are strikingly similar to those of IL-1, suggesting that IL-1 secretion, which accompanies many infections, may mediate the Nor Epinephrine and 5-HT metabolism and brain tryptophan responses, possibly via the Serotoninergic receptor and IL1 activation. Dunn A. J., Clin Neurosci. Res. 6(1-2):52-68 (2006).

Accordingly, the SGEF protein has multiple pathways available to affect fever, any one of them likely involving an effect on a cellular receptor site or a second messenger agent or possibly the control of leukocyte transendothelial migration.

Furthermore, the defective immune response in part explains the severity of the liver damage. Furthermore, however, the SGEF protein also has a role in liver homeostasis. Dysregulation is an effect of the null allele SGEF mutation. SGEF is highly expressed in the liver (more than in other tissues). See Ellerbroek, S. M. et al, Mol Biol. Cell 15:3309-3319 (2004). Therefore, the unusually extensive damage of the liver upon EBV infection points out to a role for SGEF in liver homeostasis. (No limitation of the invention in respect to the mechanism of action are implied by these observations by the inventor.)

Child 1 was shown to carry the same SGEF gene homozygous deletion as Child 2 but has no defects in the ABC4 gene. Albeit his vision and OCT tests were normal, multifocal electroretinogram (ERG) (which records retinal electrical activity of the central part of the retina using corneal, frontal and temporal electrodes during light stimulation of each eye) (focused on fovea, the center of the visual axis) showed a severely dysmorphic poorly developed fovea bilaterally. No liver study was performed on Child 1.

Accordingly, albeit Child 2 had two isoform variants in the ABC4 locus, the macula development defect was at least in part caused by the SGEF defect, as Child 1 had the macular structural defect but no ABCA4 mutations. Furthermore, the fovea and CC abnormality were seen in both of the two children having a common homozygous gene condition defect.

The father is heterozygous for the 3q25.2 deletion and had the two ABCA4 locus variant isoforms. The MRI results were normal for corpus callosum, and the hippocampus. Mutifocal ERG revealed the fovea of one eye was affected, with significantly reduced foveal cone function. The observations that SGEF defects lead to deficiency in foveal cone function is consistent with a conclusion that SGEF is responsible for neuronal and possibly blood vessel guidance—when SGEF protein is absent, the neuron and/or blood vessel deviate in their growth path, invade the fovea and interfere with cone formation and/or function. The effect is seen even in a heterozygous individual for the gene defect. Cell surface receptors like the ephrin receptor tyrosine kinase at the surface of neurons have been shown to activate the GTPase RhoA via the Rho GEF ephexin to cause myosin-dependent contraction of the actin filament cytoskeleton and to thus cause growth cone collapse of the axon tip. Alberts B. et al, supra, at page 921-22. The father was also shown by erg multifocal analysis to have defective foveal cone function unilaterally. (No limitation of the invention with respect to the mechanism of action is implied by these observations by the inventor.)

The mother, who is heterozygous for the 3q25.2 locus deletion was not shown to harbor defects in the hippocampal or CC development, but had granular ocular fundi. Again, a heterozygous individual was nonetheless at least partially affected.

These cumulative observations on this family are summarized in Table 1. (In Table 1, ND stands for no defect found. NT stands for not tested.)

TABLE 1

| | | | | Child 2 - | |
|---|---|---|---|---|---|
| | Mother | Father | Child 1 | proband | Child 3 |
| 3q25.2 locus | Hetero- | Hetero- | Homo- | Homo- | Homo- |
| deletion | zygous | zygous | zygous | zygous | zygous |
| ABCA4 | ND | Two | ND | Tested, same | ND |
| locus | | variant | | two isoforms | |
| variant | | isoforms | | as in father | |
| isoforms | | present. | | are present. | |
| RP genes, 18 | NT | NT | NT | no defects | NT |
| loci | | | | observed. | |
| Vision, | Granular | Fovea | Fovea | Multiple | NT |
| macula, | texture of | of one | of both | functional | |
| fovea, | fundi | eye is | eyes de- | defects and | |
| development | | defective | fective | fovea and | |
| and function. | | on Mf | on Mf | fundus | |
| | | ERG | ERG, | structural | |
| | | | | defects. | |
| CC | ND | ND | MRI | Complete | NT |
| develop- | | | revealed | absence, | |
| ment, func- | | | small | reduced axon | |
| tion and | | | defect. | white matter | |
| structure | | | | | |
| Hippocampal | ND | ND | ND | Hypoplasia, | NT |
| development | | | | reduced gray | |
| and function | | | | matter | |
| Immune | NT | NT | NT | Streptococcus | NT |
| function and | | | | A and EBV | |
| fever re- | | | | infections; | |
| sponse | | | | Lack of fever | |
| Liver | NT | NT | NT | Severe liver | NT |
| homeostasis | | | | damage. | |

Accordingly, although there are differences in the severity of the conditions, the SGEF has a mediating role in proper development of the macula and in particular the fovea, the CC, the hippocampal region and immune response and liver homeostasis, a role observed in both homozygous and heterozygous individuals.

A series of patients were tested for the 3q25.2 deletion including the family reported by Descartes et al., supra, who described a brother and sister with non-documented Stargardt's disease and CCA (but with more severe handicap involving facial dysmorphism, mental retardation and deafness). A second family with retinal dystrophy, CCA and mental retardation was also tested. Using DNA sequencing and quantitative PCR, both families were shown to be negative for SGEF involvement. A 100 patient cohort affected with Aicardi syndrome and other CCA patients, various macular dystrophy phenotypes, ABCA4 mutation-negative Stargardt disease patients as well as Age-related Macular Dystrophy (AMD) patients were genotyped using sequencing, but no SGEF mutations were identified. Therefore, the 3q25.2 SGEF gene is not the only gene locus responsible for syndromes affecting the retinal macular, corpus callosum and hippocampal development and immune function. Nonetheless, insufficient SGEF also has a negative role in the development of these systems and functions.

The macular foveal development is conditioned by the lack of blood vessel entry and highly dense cone photoreceptor enrichment, critical to the spatial resolving power of the fovea, where cone inner segment spacing reaches a peak of 100,000 to 300,000 mm⁻². Curcio C. A. et al, J Comp Neurol. 292:497-523 (1990).

Without limiting the invention to a particular mechanism of action, it should be noted that a method of interaction between SGEF and Phosphoinositide 3-kinase (PI3K) is apparent. In particular, PI3K constitutes docking sites for the plekstrin homology (PH) domain of SGEF. PI3K is a lipid kinase that phosphorylates phosphatidyl inositides in lipid bilayer membranes. The role of the SGEF deletion in causing macular cone dysfunction is therefore supported by the recent finding that cone dystrophy has been described in association with PI3K deficiency in mice PI3K is a classic survival kinase linking extracellular trophic/growth factors with intracellular anti-apoptotic pathways Ivanovic, I. et al, Invest. Ophthalmol. Vis. Sci., 2011, March [Epub ahead of print] PMID:21398281.

Provis, J. M. et al, Association for Research in Vision and Ophthalmology annual meeting, poster 4014/A125 (2009) discussed whether the critical lack of development of blood vessels in the macula is due to the role of axon guidance genes controlled by an interaction with netrin-UNC5 or Ephrin-6 repelling their growth in the macula and particularly into the fovea or only due to anti-angiogenic factors. Ephrin 6A seems to be present in the ganglion cell layer of fetal macaque retina in incremental axial Posterior to anterior gradient concentration to the fovea thus repressing entry of endothelial cells and blood vessels into the foveal region of the retina according to Provis et al, Id. The presence of Ephrin6A (a neuronal guidance gene) gradient would thus be a factor that blocks blood vessel entry into the retina as could be discussed with regards to SGEF (which is clearly also playing a role in neuronal guidance as evidenced by the fact that its deficiency causes ACC).

The situation where a congenital macular anomaly is a sign of a developmental defect presenting as an early onset macular dystrophy is linked to complete lack of function of SGEF in the fetal retina, which thus indicates a role for this gene in embryonic macular development.

The association of corpus callosum agenesis in the homozygous null allele is consistent with a role for SGEF in axon guidance at the level of the interhemispheric fissure interacting with the L1CAM gene product cited above and possibly the HESX1gene product (which controls the septum pellucidum (a white matter midline brain structure) formation) or BMP Bone Morphogenic Protein signaling which have all been shown to mediate Corpus callosum formation. Paul, supra. The external hydrocephalus observed in the SGEF homozygous null allele is more evidence pertaining to the role in axon guidance in meningeal development because the outer brain meninges are the site of the external brain fluid control.

During fetal brain development, axons growing from pyramidal neurons of cortical layer III extend and cross the midline. In experimental models, e.g. mice, it is possible to decipher two conditions in which the development of the corpus callosum is impaired. The first condition is characterized by an impairment of the formation of the roof of the telencephalon (the primordium of the commissural plate). This condition can be explained by an abortive induction of this region by an impairment of BMP signaling. This can generate all the forms of holoprosencephaly. Other forms are due to a defective gene coding Hesx1, a transcription factor involved in the control of telencephalic morphogenesis. Such a genetic defect in HESX1 can be observed in human dominant forms of septo-optic dysplasia. The second condition is explained by an impairment of the molecular control of axon growth: such is the case for the couple netrin 1 and DCC or for the adhesion molecule L1CAM.

Other genes originally identified by their involvement in axon patterning are also implicated in vascular patterning. The semaphorin-plexin family of genes share with VEGFA the capacity to bind neuropilin 1, expressed by both blood vessels and axons. Class 3 semaphorins are also known to have a repellent effect during vascular morphogenesis via interactions with integrins. Serini, G. et al, Nature 424:391-7 (2003). Eph receptors and their ephrin ligands have key roles in axon guidance, provide guidance cues for endothelial cells during development, are involved in assembly and maintenance of vascular networks, and arteriovenous differentiation. Pfaff, D. et al, J Leukoc Biol. 80:719-26 (2006). Netrin is a potent vascular mitogen and has a role in repelling developing vessels via interactions with the UNC5 receptor, while Slit2 is implicated in endothelial cell migration Park, K. W. et al, Proc Natl Acad Sci USA 101:16210-5 (2004); Suchting, S. et al, Exp Cell Res. 312:668-75 (2006). Of particular interest are the repellent effects of netrin-UNC5 interactions and Eph-ephrin signaling on developing vessels as well as axons during development Lu, X. et al., Nature 432:179-86 (2004); Cowan C. A. et al., Trends Cell Biol. 12:339-346 (2002). Accordingly, we conclude that a graded expression of genes involved in repellent signaling that is centered on the fovea during development—similar to the one reported for Eph-A6-retards the growth of vessels into the central region of the retina, and contributes to definition and developmental pattern of the foveal avascular area.

It has been shown that bacterial pathogens such as Salmonella Typhimurium use RhoG activation to enter the host cell. Patel J. C. and Galán, J. E., J Cell Biol. 175(3):453-63, Epub (2006). The authors performed an RNA interference screen for Rho GTPases that could account for SopB-dependent invasion. They found that knockdown of RhoG resulted in reduced levels of serovar Typhimurium invasion. RhoG was activated and recruited to sites of serovar Typhimurium invasion in a SopB-dependent manner. Next, they investigated how SopB activates RhoG and discovered that SGEF (SH3-containing guanine nucleotide exchange factor) was recruited to ruffles in a SopB-dependent manner and that it was required for SopB-dependent RhoG activation. This is a key evidence of the role of SGEF in bacterial infectious disease.

The SGEF gene/protein are also known by other names, i.e. cSGEF for a terminal 3′ isoform; HMFN1864; DKFZp434D146; and ARHGEF26. See, [http://www.ncbi.nlm.nih.gov/pubmed?Db=gene&Cmd=retrieve&dopt=full_report&list_uids=26084], last viewed on Jan. 31, 2011. Diversification of transcriptional modulation: large-scale identification and characterization of putative alternative promoters of human genes, Kimura, K. et al, Genome Res. 16(1):55-65 (2006); Expression profiling and differential screening between hepatoblastomas and the corresponding normal livers: identification of high expression of the PLK1 oncogene as a poor-prognostic indicator of hepatoblastomas, Yamada, S. et al, Oncogene, 23(35):5901-11 (2004); SGEF, a RhoG guanine nucleotide exchange factor that stimulates macropinocytosis, Ellerbroek S M, et al., Mol Biol Cell, 15(7):3309-19 (2004); Ota T. et al., Nat Genet. 36(1):40-5 (2004); Isolation of the novel human guanine nucleotide exchange factor Src homology 3 domain-containing guanine nucleotide exchange factor (SGEF) and of C-terminal SGEF, an N-terminally truncated form of SGEF, the expression of which is regulated by androgen in prostate cancer cells, Qi H. et al., Endocrinology, 144(5):1742-52 (2003), and Generation and initial analysis of more than 15,000 full-length human and mouse cDNA sequences, Strausberg R. L. et al., Proc Natl Acad Sci U.S.A. 99(26):6899-903 (2002).

A summary of the gene information is found in Thierry-Mieg, Danielle and Thierry-Mieg, Jean, Genome Biology 7(1):512 (2006), or online at http://www.ncbi.nlm.nih.gov/IEB/Research/Acembly/ay.cgi?db=human&1=SGEF, last viewed on Feb. 10, 2011.

The complete gene sequence is available on Genebank under accession number AC_{—}000046.1, using the GRCh37.p2 primary reference assembly:

http://www.ncbi.nlm.nih.govinuccore/NC_{—}000003.11?from=153839149&to =153975 616&report=genbank, last viewed on Mar. 7, 2011.

The gene is ubiquitously expressed and is expressed at higher than average levels. It has very high expression in retina, brain and liver. As noted above, the gene sequence information as well as the location of exons and introns are known. The sequences of mRNAs isolated from various tissues are also known. Deduced amino acid products are provided. Alternative SGEF protein variants are produced, depending on alternative expression and processing, e.g. splicing and choice of transcriptional promoter. There is sufficient information to allow an artisan skilled in the art using known methods to construct an artificial gene and vector for expression of an SGEF protein and variants.

In accordance to one aspect of the invention, an SGEF protein is provided to a mammalian patient. Preferably, more than one SGEF protein is provided to a mammal. Alternatively, an artificial SGEF gene may be expressed in a mammal or the genes encoding variants may be co-expressed in the mammal. Homologs of SGEF are also within the scope of the invention. Homologs of SGEF are SGEF proteins from species other than Homo sapiens. All possible protein constructs based on the SGEF gene sequence (larger than about 90 amino acids, up to about 900 amino acids) or from the prototype sequences listed below, are within the scope of this invention.

According to a preferred embodiment, as an example the artificial SGEF gene encodes a protein which is 751 amino acids (“aa”) in length. The preferred prototype 751 aa protein sequences is:

(SEQ ID NO 1.)
MDGESEVDFSSNSITPLWRRRSIPQPHQVLGRSKPRPQSYQSPNGL
LITDFPVEDGGTLLAAQIPAQVPTASDSRTVHRSPLLLGAQRRAVA
NGGTASPEYRAASPRLRRPKSPKLPKAVPGGSPKSPANGAVTLPAP
PPPPVLRPPRTPNAPAPCTPEEDLTGLTASPVPSPTANGLAANNDS
PGSGSQSGRKAKDPERGLFPGPQKSSSEQKLPLQRLPSQENELLEN
PSVVLSTNSPAALKVGKQQIIPKSLASEIKISKSNNQNVEPHKRLL
KVRSMVEGLGGPLGHAGEESEVDNDVDSPGSLRRGLRSTSYRRAVV
SGFDFDSPTSSKKKNRMSQPVLKVVMEDKEKFSSLGRIKKKMLKGQ
GTFDGEENAVLYQNYKEKALDIDSDEESEPKEQKSDEKIVIHHKPL
RSTWSQLSAVKRKGLSQTVSQEERKRQEAIFEVISSEHSYLLSLEI
LIRMFKNSKELSDTMTKTERHHLFSNITDVCEASKKFFIELEARHQ
NNIFIDDISDIVEKHTASTFDPYVKYCTNEVYQQRTLQKLLATNPS
FKEVLSRIESHEDCRNLPMISFLILPMQRVTRLPLLMDTICQKTPK
DSPKYEVCKRALKEVSKLVRLCNEGARKMERTEMMYTINSQLEFKI
KPFPLVSSSRWLVKRGELTAYVEDTVLFSRRTSKQQVYFFLFNDVL
IITKKKSEESYNVNDYSLRDQLLVESCDNEELNSSPGKNSSTMLYS
RQSSASQSPLYSDSP*

(In protein sequences “*” denotes a stop codon is present at this location of a coding sequence.) Another example of an SGEF protein prototype is about 446 aa in length. A 446 aa protein preferably has the following sequence:

(SEQ ID NO 2.)
MDGESEVDFSSNSITPLWRRRSIPQPHQVLGRSKPRPQSYQSPNGL
LITDFPVEDGGTLLAAQIPAQVPTASDSRTVHRSPLLLGAQRRAVA
NGGTASPEYRAASPRLRRPKSPKLPKAVPGGSPKSPANGAVTLPAP
PPPPVLRPPRTPNAPAPCTPEEDLTGLTASPVPSPTANGLAANNDS
PGSGSQSGRKAKDPERGLFPGPQKSSSEQKLPLQRLPSQENELLEN
PSVVLSTNSPAALKVGKQQIIPKSLASEIKISKSNNQNVEPHKRLL
KVRSMVEGLGGPLGHAGEESEVDNDVDSPGSLRRGLRSTSYRRAVV
SGFDFDSPTSSKKKNRMSQPVLKVVMEDKEKFSSLGRIKKKMLKGQ
GTFDGEENAVLYQNYKEKALDIDSDEESEPKEQKSDEKIVIHHKPL
RSTWSQLSAVKRKVILIVGFMEMKDGRLRGGK*

Another example of an SGEF protein prototype is at least about 110 aa in length, likely longer. The at least 110 aa protein prototype preferably has the following sequence:

(SEQ ID NO 3.)
MDGESEVDFSSNSITPLWRRRSIPQPHQVLGRSKPRPQSYQSPNGLLI
TDFPVEDGGTLLAAQIPAQVPTASDSRTVHRSPLLLGAQRRAVANGGT
ASPEYRAASPRLRR
(An incomplete sequence, the mRNA does not
comprise a stop codon at this location.)

It will be noted that the above prototype sequences comprise the same amino acid sequence at their N-termini.

Another example of an SGEF protein is about 154 aa in length. A 154 aa protein prototype would preferably have the following sequence:

(SEQ ID NO 4.)
MKSLILLQGRTAPQCSIQDRALPVSHLFTLTVLSNHANEKVEMLLGA
ETQSERARWITALGHSSGKPPADRTSLTQVEIVRSFTAKQPDELSLQ
VADVVLIYQRVSDGWYEGERLRDGERGWFPMECAKEITCQATIDKNV
ERMGRLLGLETNV*

Yet another example of an SGEF protein is about 137 aa in length. A 137 aa protein prototype would preferably have the following sequence:

(SEQ ID NO 5.)
MFCFLLEAQLVSLNAGPELQRKISKCTLLDCTCFFSATGNLVCPLLA
SALTQVEIVRSFTAKQPDELSLQVADVVLIYQRVSDGEWERSYGTLV
VQDAECYRPEECHFVIIAHIPNLDMLMFEITYMYCLLISKAKP*

It will be noted that the protein sequences of SEQ. ID. NOs. 4 and 5 comprise an overlap region. Therefore, the protein of the invention is any of the above illustrated protein prototypes, as well as any other protein construct based on the SGEF gene sequence, which is at least about 90 amino acids, up to about 900 amino acids long. Such proteins result, for example, from alternative transcriptional promoters, alternative processing of the transcripts and/or alternative protein processing possibly mediated by specific 5′ region enhancers or repressors.

The SGEF protein of invention does not have to be identical to a naturally derived SGEF protein, it can be a variant protein. Two amino acid sequences are said to be “identical” if the two sequences, when aligned with each other, are having exactly the same amino acid sequences, with no gaps, substitutions, insertions or deletions. The variant proteins of the invention are, preferably, identical to one of the prototype amino acid sequences identified by SEQ ID NOs 1-5.

However, the proteins of the invention do not have to be identical to any of these sequences. The scope of the invention includes protein variants having sequences that are “substantially identical” (as defined below) to one of the sequences identified by SEQ ID NOs 1-5, or to any protein based on the SGEF gene sequence.

The protein sequence of the invention may comprise acceptable substitute amino acids. Certain amino acids are “like” amino acids in certain aspects, e.g. size, shape, and polarity. “Like” amino acids substitutions and their use as substitutes are concepts well understood in the art. By way of example, glycine, alanine, serine, threonine and methionine are considered to be “short side chain” amino acids; isoleucine, leucine and valine are all hydrophobic in nature; asparagine and glutamine are polar; aspartic acid and glutamic acid are acidic; lysine, arginine and histidine are basic; and tyrosine phenylalanine, and tryptophan have aromatic group shaped side chains. Such “like” substitutions do not likely have a significant impact on the protein's folding and function. In accordance to the invention, if the like-substitutions do not amount to changes in amino acid identity at the corresponding position in more than 60% of the protein sequence, the protein is an SGEF protein of the invention. Preferably, the like-aa substitution comprises less than about 60% of the sequence, more preferably about 50%, or 45%, or 40%, yet more preferably, about 35%, 30%, 25%, 20%, or 15% and more preferably yet, about 10%, 5% or about 0% like-amino acid substitutions.

In respect to the proteins identified by SEQ ID NOs 1-3, certain substitutions including V29L; L605; F2035; L461M, 5707T; Q743H and S744L; P745F are “acceptable substitutions” and their presence does not contribute to the above calculation of allowable substitutions Likewise, substitutions including S25T; H26S and F28L are acceptable substitutions of the prototype sequence of SEQ ID No 4.

Single nucleotide polymorphisms (SNPs), have been frequently involved in controlling the level of expression of the gene in different tissues and to thus mediate predisposition to as well as protection from different disorders. Such SNP variants have been implicated in multiple disorders from breast cancer to diabetes and age-related macular degeneration. SNP variants of SGEF are important factors in mediating visual capacity via macular function, bi-manual and hand-eye coordination and speed via corpus callosum development, liver homeostasis and sensitivity to drugs, alcohol or toxic substances, the immune function relative to viral or bacterial pathogens and mounting of an inflammatory response manifesting as fever, interferon, interleukin and other inflammatory mediator synthesis or secretions. A SGEF protein variant, wherein the amino acid sequence is modified in correspondence to an SNP, are considered SGEF proteins desirable as therapeutic agent of diseases that correlate with development and function of the retinal macula, the corpus callosum, the hippocampus, the liver, the immune system, and is a factor in a fever response to infections Likewise, the SNP might beneficially reduce the level of gene expression. For example, it can reduce the likelihood of cancer, of inflammation and of atheriosclerosis by blocking transendothelial migration. Thus any natural variant of the SGEF gene or portion thereof can be advantageously expressed in a patient. The natural variant expressed is preferably a variant comprising a SNP variation in a functional domain of an SGEF protein. More preferably, the SNP causes a change in the primary structure of a protein domain such as the DH domain and the PH domain or the SH3 domain.

Moreover, a protein sequence need not be perfectly aligned to another sequence and be expected to retain functionality. Short gaps and additions are tolerated. A preferred protein of the invention has the same overall length as a respective prototype listed in one of the sequences of SEQ ID NO 1-5, but the sequence may be up to about 15% different in length, as long as any one deletion or insertion does not comprise more than 15 consecutive amino acid residues. Preferably, the difference in length of the sequences is about 12%, 10% or 8%. More preferably, the length differences are about 7%, 6%, 5%, 4%, 3%, 2%, or 1%. Preferably, an addition or deletion is no more than about 12, 10, 8, 7, 5, 3, or 2 consecutively strung out aa residues.

Moreover, the protein(s) of the invention must comprise at least one and preferably more than one of the functional domains listed below. GEF stands for guanine nucleotide exchange factor which is the rate limiting step of the GTPase cycle and SGEF indicates the protein also includes a C terminal SH3 protein domain, flanking the hinge and binding specificity loops which binds to proline-rich ligands. It is also referred to as the SRC Homology 3 domain. The SH3 is a small protein domain of about 60 amino acids residues. It has been identified in several other protein families such as: tyrosine kinases, phosphotases and cytoskeletal proteins like myosin 1, spectrin and contactin; PI3 Kinase, Ras GTPase activating protein (Ras-GAP), the GEF VAV, Crk adapter protein, CDC24 and CDC25. The SH3 domain has a characteristic beta-barrel fold which consists of five or six 13-strands arranged as two tightly packed anti-parallel 0 sheets. The linker regions may contain short helices. The SH3 domain is usually found in proteins that interact with other proteins and mediate assembly of specific protein complexes like scaffolds, typically via binding to proline-rich peptides (specifically left handed type 1 polyproline helices that repeat every 3 residues in their respective binding partner. Many SH3-binding epitopes of proteins have a consensus sequence:

-X-P-p-X-P-
-1-2-3-4-5-

with 1 and 4 being aliphatic amino acids, 2 and 5 always and 3 sometimes being proline. The sequence binds to the hydrophobic pocket of the SH3 domain. Interaction depends on hydrophobic contacts of proline with conserved hydrophobic residues in a shallow groove on the SH3 domain as well as hydrogen bonds with ligand peptide carbonyl oxygen. SH3 domains that bind to a core consensus motif R-x-x-K have been described. Examples are the C-terminal SH3 domains of adaptor proteins like Grb2 and Mona (a.k.a. Gads, Grap2, Grf40, GrpL etc.). Other SH3 binding motifs have emerged and are still emerging in the course of various molecular studies, highlighting the versatility of this domain. Preferably, the proteins of the invention similar to prototypes identified by SEQ ID NOs 1 and 2 and contain the SH domain.

TABLE 2

| Location | Cyto | | | |
|---|---|---|---|---|
| of the | genetic | Probes (from | Startsite | Stopsite |
| Probe | Band | Affymetrix) | location | location |
| Last normal | 3q 25.2 | A_14_P137770 | 155170885 | 155170944 |
| First | 3q 25.2 | A_16_P16459148 | 155239440 | 155239499 |
| deleted | | | | |
| Last | 3q 25.2 | A_14_P136564 | 155353325 | 155353384 |
| deleted | | | | |
| Next | 3q 25.2 | A_16_P16459487 | 155368151 | 155368210 |
| normal | | | | |

The minimal size of the deletion is 113944 base pairs and its maximum size is 197207 base pairs. Accordingly, the gene transmitted within the family of the Example 1 is an SGEF gene. The syndrome was caused by a homozygous deletion within that gene.

### Example 4

### The Segregation Analysis of Example 1 and the Identification of the Gene Defect Lead to Conclusions as to the Role of SGEF Gene

From the segregation analysis of Example 1 and the gene mapping data of Example 3, we conclude that the SGEF homozygous deletion is sufficient to also cause subclinical phenotypes like the bilateral foveal macular dysfunction and minimal corpus callosum development defect seen in the brother who does not harbor the double ABCA4 variant. The cumulative effect of SGEF homozygous deletion with the double ABCA4 variant possibly causes the added phenotype observed in the proband with congenital nystagmus with macular dystrophy but it could also be linked to SGEF effect alone and other causes like epigenetic factors or other genetic factor. Indeed the non ocular phenotype seen in the proband with complete agenesis of the corpus callosum, the hippocampal hypoplasia and the immune dysfunction is difficult to attribute to such mild effect ABCA4 variants. Bhongsatiern J, et al, J. Neurochem. 92(5):1277-1280 (2005); Tachikawa M, et al, J. Neurochem. 95(1):294-304 (2005); Warren M S et al, Pharmacol Res. 59(6):404-13 (2009); and Tsybovsky Y. et al, Adv Exp Med Biol. 703:105-25 (2010).

Therefore the variable severity of the ocular and brain phenotype between proband and older sib could possibly be due to the redundancy of the GTPase pathway. The single SGEF deletion in the father gives a unilateral subclinical foveal macular dysfunction despite the presence of the ABCA4 variant while the double deletion in the brother gives a more marked but bilateral subclinical phenotype in the brother who does not harbor the ABCA4 variant. This indicates a strong role of the SGEF homozygous deletion in the foveal macular defect as well as the CCA. Another clinical observation is the fact that on angiography the proband does not show the increased auto-fluorescence typical of ABCA4 Stargardt's disease due to A2E deposits giving rise to the lipofuscin deposits. This evidence goes against the ABCA4 defect as a major cause of the macular dystrophy.

Accordingly, albeit other gene loci may contribute to macular as well as corpus callosum defects, the role of SGEF is clearly established.

It has been shown, above, that SGEF has a key role in retinal macula, corpus callosum, hippocampus, liver and immune systems function and structure. Albeit the invention is not limited by the mechanism of action, it is of interest to note that these activities might involve SGEF's role as an activator of Rho GTPases. Some of these effects are mediated by the interaction with the actin cytoskeleton. Other effects might be initiated by receptor tyrosine kinases or Gprotein coupled receptors. SGEF is a key to modulation of Rho GTPase signaling which is a hub to promote normal neuronal connectivity and its regulation in response to extracellular signals and environment. While we have shown the key role of SGEF in promoting normal healthy inflammatory immune response and fever, its overexpression can be detrimental.

The scope of the invention also includes control of diseases caused by over-expression of SGEF or by excessive inflammation. The over-expressed SGEF might interact with the cytoskeleton to mediate cell movement, or the over-expressed SGEF might affect cell-cell interactions, transendothelial migration, cell division and multiplication, as well as cell transformation.

Because of its role in transendothelial migration SGEF can be understood as mediating a key step of the scavenging and prevention of atherosclerosis and plaques. Hagg, S. et al, PLoS Genet. 5(12):e1000754 (2009). (Epub 2009.); Van Buul JD et al, Arterioscler ThrombVasc Biol 24:824-833 (2004). Rho GTPases activity plays a key role in this process. Rolfe BE et al, 183(1):1-16 (2005). Epub 2005, Jun. 27.) Thus, controlling/modulating SGEF, systemically, locally, or temporarily is a useful tool for the prevention and treatment of atherosclerosis.

Modulating SGEF must take into consideration its effects in multiple situations and the specific facts of a particular patient. For example, as noted above, SGEF has a role in activation of RHO GTPases and thus affect inflammatory cells like macrophage migration and phagocytosis, lipid uptake, a role in endothelial cells via PI3K intracellular signal transduction and also a role in vascular smooth cells in proliferation/migration and extracellular matrix uptake. It is the combination of these factors that contribute to endothelial dysfunction, coronary vasospasm, intimal hyperplasia and atherosclerosis.

However, the SGEF effect differs on the various systems. By way of example, both the inflammation response and the atherosclerosis mechanism involve active or overly active macrophages. As noted above, it was now observed that an SGEF deficient patient lacks an appropriate inflammatory response. This evidences a role for SGEF in normal macrophage function. Without limiting the invention to a particular mechanism of action, the absence of fever is due to a lack of transendothelial migration of macrophage or lack of macrophage activation or phagocytosis. Accordingly, increased SGEF activity or expression is recommended for treatment of the patient lacking an adequate inflammatory response. A more controlled (reduced) macrophage activation would, on the other hand, lower the process of plaque formation in atherosclerosis. Accordingly, a somewhat diminished SGEF expression or activity level and a corresponding reduction in macrophage activation is beneficial to a patient prone to atherosclerosis, e.g. an obese patient, a patient with hypercholesterolemia or a diabetic.

Appropriate SGEF levels are critical for prevention and for control of multiple phenomena and a balance must be considered, under specific facts. For example, consider inflammation and atherosclerosis. Depending on the patient's profile, diagnosis and stage of disease development, one may choose to increase or decrease the level of SGEF activity. In severe cases of atherosclerosis, reduced SGEF activity levels are desirable. Generally an about 3% to about 80% reduction in the SGEF level is desirable, reduction by about 10% to about 50% yet more desirable, and a reduction by about 20% to about 50% more desirable yet. In a patient lacking a desirable inflammatory response, stimulation of SGEF is desired, in a controlled, perhaps temporary manner.

It has also recently been shown that low expression of SGEF is a marker of poor response to chemotherapy in ovarian cancer cells. Kim, S. W. et al, OMICS 2011, Feb. 19 [Epub ahead of print].

Because of the pivotal role of SGEF in cell multiplication and growth, we conclude that the prolonged excess of SGEF gene expression or activation is a factor in carcinogenesis and cell transformation. Inhibitors of SGEF play a role in cancer treatments of prostate and other cancers.

Another line of evidence is provided by inhibition of Rho kinase which is a downstream effector of GTPases as an effective tool to block cell migration Tsai CC. et al, Biochem Pharmacol. 2011 Jan. 26. [Epub ahead of print]. This key property is another line of evidence that SGEF inhibitors are a useful treatment to block tumor cell migration.

Similarly gain of function mutations in the Ras GEF named SOS (“son of sevenless”) cause Noonan syndrome where cancer is a frequent complication. (Roberts, A. E. et al, Germline gain-of-function mutations in SOS1 cause Noonan syndrome. Nature Genet. 39: 70-74, (2007) and Tartaglia, M. et al., Nature Genet. 39: 75-79 (2007).

Rho kinase (ROCK1 and ROCK2) is a serine/threonine kinase that serves as a downstream effector of Rho GTPase. This class of kinases plays a key role in regulating the contractile tone of smooth muscle tissue through actin stress fibers via the phosphorylation of MLC (myosin light chain) in a calcium-independent manner. Myosin phosphorylation and the resultant increase in the contractile state are regulated through ROCK and subsequent vascular smooth muscle mediators such as Nitric oxide and endothelin. Rock inhibition leads to the relaxation of smooth muscle fibers. Experimental evidence indicates that inhibiting ROCK activity through topical and systemic ROCK inhibition could be beneficial for the treatment of increased intraocular pressure in patients with glaucoma, because both ROCK and Rho GTPase inhibitors can increase aqueous humor drainage via trabecular meshwork smooth muscle and ciliary muscle relaxation.

In ocular trabecular meshwork (TM) cells, the primary function of membrane-anchored Rho G-proteins is to promote filamentous actin stress fiber organization. Tian B. Exp Eye Res. 88:713-717 (2009). Activation of RhoG signaling enhances the contractile tone of TM cells, leading to slower rates of aqueous humor (AH) outflow and higher intraocular pressure (TOP). (Rao V P & Epstein D L. Biodrugs. 2007; 21:167-177.) Inhibition of RhoG proteins or downstream RhoG effectors, such as Rho kinase, enhances AH outflow facility, thereby reducing IOP. Consequently, selective inhibitors of Rho signaling are aggressively being explored as potential therapeutic agents for the management of ocular hypertension (Von Zee C L. & Stubbs, E B. Jr. IOVS March 2011 52:1676-1683; published ahead of print Jan. 6, 2011, doi:10.1167/iovs.10-6171)

In a parallel manner, another upstream activator of Rho G, the SGEF protein or protein expression inhibitor also is used as a therapeutic agent of increased intraocular pressure. Since SGEF is a strong activator of RhoG we conclude that inhibition of SGEF in the anterior segment of the eye is useful as a smooth muscle and ciliary muscle relaxant and therefore a useful glaucoma or elevated intraocular treatment either topically or systemically.

An oral ROCK Inhibitor, Fasudil, is used in Japan for the prevention of cerebral vasospasm in patients with subarachnoid hemorrhage. (Lau C. et al, Br J. Pharmacol. 2011 Feb. 10. doi: 10.1111/j.1476-5381.2011.01259. Epub ahead of print]). Ditto, another upstream regulator of rho GTPase, the SGEF protein or protein expression inhibitor also is used as a therapeutic agent of increased intraocular pressure or glaucoma.

Fasudil along with other ROCK Inhibitors have been shown to reverse vasoconstriction, alter and improve blood flow after ischemic reperfusion injury, have neuroprotective properties, inhibit cellular proliferation, and inhibit inflammation. Preclinical models specific to cerebral and ocular injury are suggestive that ROCK Inhibitors could improve Retinal Ganglion Cell survival and axon regeneration, thus providing a potential benefit to patients with glaucomatous injury beyond IOP reduction. Local SGEF inhibition could have similar neuroprotective effects of retinal ganglion cells in glaucoma as well as ischemic reperfusion injury.

Rho kinase inhibition decreases liver fibrosis and SGEF inhibition has a similar effect of preventing liver fibrosis if delivered directly to the liver, for example as a conjugate to Glucose 6 phosphate human serum albumin which is selectively taken up by stellate liver cells. Van Beuge M et al, J Pharmacol Exp Ther. 201. [Epub ahead of print].) A SGEF inhibitor will thus have a protective effect against liver fibrosis.

Rho kinase inhibition has also been associated with treatment of osteoarthritis in animal models Takeshita N, J Pharmacol Sci. 2011 Feb. 16. [Epub ahead of print]) and thus an SGEF inhibitor is useful as a preventive and curative treatment of osteoarthritis and joint inflammatory processes, locally and systemically.

### ADDITIONAL REFERENCES

1. Saraste, M. and Hyvonen, M. “Pleckstrin homology domains: a fact file” Curr. Opin. Struct. Biol. 5(3):403-408 (1995).
2. Riddihough, G. “More meanders and sandwiches” Nat. Struct. Biol. 1(11):755-757 (1994).
3. [[2]. Hart M J, Eva A, Evans T, Aaronson S A, Cerione R A Catalysis of guanine nucleotide exchange on the CDC42Hs protein by the db1 oncogene product.; Nature. 354:311-314 (1991)
4. [3]. Tan E C, et al. The human active breakpoint cluster region-related gene encodes a brain protein with homology to guanine nucleotide exchange proteins and GTPase-activating proteins.; J Biol Chem. 268:27291-27298 (1993).
5. [4]. Soisson S M, Nimnual A S, Uy M, Bar-Sagi D, Kuriyan J; Crystal structure of the Db1 and pleckstrin homology domains from the human Son of sevenless protein. Cell; 95:259-268 (1998).
6. Ridley, A. J. “Rho GTPases and actin dynamics in membrane protrusions and vesicle trafficking”. Trends Cell Biol. 16(10):522-9 (2006).
7. RHOG ras homolog gene family, member G (rho G) [Homo sapiens] Gene ID: 391, updated on 6 Mar. 2011 http://www.ncbi.nlm.nih.gov/sites/entrez ?Db=gene&Cmd=ShowDetailView&TermT oSearch=391, last viewed on Mar. 6, 2011.
8. Vincent S. et al. Growth-regulated expression of rhoG, a new member of the ras homolog gene family. Mol. Cell Biol. 12(7):3138-48 (1992).
9. Condliffe A M et al “RhoG regulates the neutrophil NADPH oxidase”. J. Immunol. 176(9):5314-20 (2006).
10. Vigorito E. et al. “RhoG regulates gene expression and the actin cytoskeleton in lymphocytes”. Oncogene 22 (3): 330-42 (2003).
11. Katoh H. et al. “Small GTPase RhoG is a key regulator for neurite outgrowth in PC12 cells” Mol. Cell Biol. 20(19): 7378-87 (2000).
12. van Buul J D, et al “RhoG regulates endothelial apical cup assembly downstream from ICAM1 engagement and is involved in leukocyte trans-endothelial migration” J. Cell Biol. 178(7): 1279-93 (2007).
13. Katoh. et al. “Activation of Rac1 by RhoG regulates cell migration” J. Cell. Sci. 119(1): 56-65 (2006).
14. Boureux A. et al. “Evolution of the Rho family of ras-like GTPases in eukaryotes”. Mol Biol Evol. 24(1): 203-16 (2007).
15. Murga C. et al. “Rac1 and RhoG promote cell survival by the activation of PI3K and Akt, independently of their ability to stimulate JNK and NF-KB”. Oncogene 21(2): 207-16 (2002).
16. Prieto-Sanchez R M, et al. “Involvement of the Rho/Rac family member RhoG in caveolar endocytosis”. Oncogene 25(21):2961-73 (2006).
17. Yamaki N, et al. “RhoG regulates anoikis through a phosphatidylinositol 3-kinase-dependent mechanism”. Exp. Cell Res. 313(13): 2821-32 (2007).
18. Blangy A, et al. “TrioGEF1 controls Rac- and Cdc42-dependent cell structures through the direct activation of RhoG”. J. Cell Sci. 113(Pt 4): 729-39 (2000).
19. Bellanger J, et al. “The two guanine nucleotide exchange factor domains of Trio link the Rac1 and the RhoA pathways in vivo”. Oncogene 16(2):147-52 (1998).
20. Estrach S, et al. “The Human Rho-GEF trio and its downstream target GTPase RhoG are involved in the NGF pathway, leading to neurite outgrowth”. Curr. Biol. 12(4): 307-12 (2002).
21. deBakker C. D., et al. “Phagocytosis of apoptotic cells is regulated by a UNC-73/TRIO-MIG-2/RhoG signaling module and armadillo repeats of CED-12/ELMO”. Curr. Biol. 14(24): 2208-16 (2004)
22. Schuebel K E, et al. “Phosphorylation-dependent and constitutive activation of Rho proteins by wild-type and oncogenic Vav-2”. EMBO J. 17(22): 6608-21 (1998).
23. Movilla N and Bustelo X R. “Biological and regulatory properties of Vav-3, a new member of the Vav family of oncoproteins”. Mol. Cell. Biol. 19(11): 7870-85 (1999).
24. Zalcman G, et al. “RhoGDI-3 is a new GDP dissociation inhibitor (GDI). Identification of a non-cytosolic GDI protein interacting with the small GTP-binding proteins RhoB and RhoG”. J. Biol. Chem. 271(48): 30366-74 (1996).
25. Cote J, Vuori K. “GEF what? Dock180 and related proteins help Rac to polarize cells in new ways”. Trends Cell Biol. 17(8): 383-93 (2007).
26. Brugnera E, et al. “Unconventional Rac-GEF activity is mediated through the Dock180-ELMO complex”. Nat. Cell Biol. 4 (8): 574-82 (2002).
27. Lu M, et al. “PH domain of ELMO functions in trans to regulate Rac activation via Dock180”. Nat. Struct. Mol. Biol. 11(8): 756-62 (2004).
28. Katoh, H. and Negishi, M. “RhoG activates Rac1 by direct interaction with the Dock180-binding protein Elmo”. Nature 424(6947): 461-64 (2003).
29. Vignal E, Blangy A, Martin M, et al. “Kinectin is a key effector of RhoG microtubule-dependent cellular activity”. Mol. Cell Biol. 21(23): 8022-34 (2001).
30. Taviaux S A, Vincent S, Fort P, Demaille JG. “Localization of ARHG, a member of the RAS homolog gene family, to 11p15.5-11p15.4 by fluorescence in situ hybridization.”. Genomics 16(3): 788-90 (1993).
31. Prieto-Sanchez R M, Bustelo X R. “Structural Basis for the Signalling Specificity of RhoG and Rac1 GTPases.”. J. Biol. Chem. 278(39): 37916-25 (2003).
32. Patel J C, Galan J E. “Investigating the Function of Rho Family GTPases during Salmonella/Host Cell interactions.”. Meth. Enzym. 439:145-58 (2008).
33. Meller N, et al. “CZH proteins: a new family of Rho-GEFs.”. J. Cell. Sci. 118(Pt 21): 4937-46 (2005).
34. Lu M, et al. “A Steric-Inhibition Model for Regulation of Nucleotide Exhchange via the Dock180 Family of GEFs.”. Curr. Biol. 15(4): 371-77 (2005).
35. Jankowski A, et al. “Capture of an activated complex from the surface of live cells by affinity receptor chromatography.”. Anal. Biochem. 380:235 (2008).
36. Vigorito E, et al. “Immunological Function in Mice Lacking the Rac-Related GTPase RhoG.”. Mol. Cell. Biol. 24(2): 719-29 (2004).
37. Meller J, et al. “Endogenous RhoG is dispensible for integrin-mediated cell spreading but contributes to Rac-independent migration.”. J. Cell. Sci. 121(Pt 12): 1981-89 (2008).
38. Miki T, et al. “Oncogene ect2 is related to regulators of small GTP-binding proteins.”. Nature 362(6419): 462-5 (1993).
39. Le Gallic L, and Fort P. “Structure of the human ARHG locus encoding the Rho/Rac-like RhoG GTPase.”. Genomics 42(1): 157-60 (1997).
40. Booden M A, Campbell S L, Der C J. “Critical but distinct roles for the pleckstrin homology and cysteine-rich domains as positive modulators of Vav2 signaling and transformation.” Mol. Cell. Biol. 22(8): 2487-97 (2002).
41. Skowronek K R, Guo F, Zheng Y, Nassar N. “The C-terminal basic tail of RhoG assists the guanine nucleotide exchange factor trio in binding to phospholipids.” J. Biol. Chem. 279(36): 37895-907 (2004).
42. Hiramoto K, Negishi M, Katoh H. “Dock4 is regulated by RhoG and promotes Rac-dependent cell migration.” Exp. Cell Res. 312(20): 4205-16 (2007).
43. Gumienny T L, Brugnera E, Tosello-Trampont A et al, “CED-12/ELMO, a Novel Member of the CrkII/Dock180/Rac Pathway, Is Required for Phagocytosis and Cell Migration”. Cell 107(1): 27-41 (2001).
44. Kunisaki Y, et al, “DOCK2 is a Rac activator that regulates motility and polarity during neutrophil chemotaxis” J. Cell. Biol. 174(5): 647-52 (2006).
45. Lu M, Ravichandran K S. “Dock180-ELMO Cooperation in Rac Activation.” Meth. Enzym. 406:388-402 (2006).

Multiple aspects of the invention were illustrated by proposing particular mechanisms of actions which appear preferred mechanisms. However, the invention's scope is not limited by a mechanism of action.

All references, including publications, patent applications, patents, and website content cited herein are hereby incorporated by reference to the same extent as if each reference were individually and specifically indicated to be incorporated by reference and was set forth in its entirety herein.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A composition comprising at least one isolated or purified, functional, SGEF protein or SGEF protein variant and a pharmaceutical carrier, prepared for introduction in a mammal.

2. The composition of claim 1, wherein said mammal has a clinical or subclinical condition for at least one disease from among a disease associated with structure or function of macula, corpus callosum, hippocampus, liver, immune function or feverless infection.

3. The composition of claim 1, comprising a variant of a SGEF protein encoded by a SGEF gene located at 3q25.2.

4. The composition of claim 1, wherein said at least one SGEF protein variant is selected from among the protein variants of SEQ ID No 1, SEQ ID No 2, SEQ ID No 3, SEQ ID No 4, or SEQ ID No 5.

5. The composition of claim 4, wherein said at least one SGEF protein variant is selected from among the protein variants of SEQ ID No 1, SEQ ID No 2, or SEQ ID No 3.

6. The composition of claim 1, wherein said SGEF protein or SGEF protein variant is introduced in said mammal as a nucleic acid molecule engineered for expression of said SGEF protein or SGEF protein variant in said mammal.

7. A method of treatment comprising providing at least one SGEF protein variant and a pharmaceutical carrier to an individual manifesting a clinical or subclinical condition or predisposition for a disease associated with functional or structural defects corresponding to a retina/macula anomaly (“RMA”), corpus callosum anomaly, hippocampus anomaly, liver disease, immune response deficiency or feverless infection.

8. The method of claim 7, wherein said disease state is associated with RMA and comprises at least one disorder from among retinal disorders, macular disorders, macular dystrophies or macular degenerations like age-related macular degeneration, geographic atrophy, diabetic retinopathy, glaucomatous retinal dysfunction and visual disorders.

9. The method of claim 8, wherein said disease state is associated with corpus callosum anomaly and comprises at least one disorder from among hypoplasia, absence or thickened corpus callosum and coordination disorders, including hand-eye coordination disorders.

10. The method of claim 7, wherein said disease state is associated with hippocampal development deficiency or dysfunction and comprises at least one disorder from among memory dysfunction, intellectual deficiency, mental retardation, Alzheimer disease or degenerative brain disorders.

11. The method of claim 7, wherein said disease state is associated with immune deficiency and comprises at least one disorder from among immune deficiency disorder caused by HIV infection, congenital immune deficiencies, ADA (adenosine deaminase), or steroid induced immune deficiency.

12. The method of claim 7, wherein said disease state is associated with liver disease and comprises at least one disease from among hepatitis, congenital liver disease, liver cirrhosis or lack of liver homeostasis.

13. The method of claim 7, wherein said SGEF protein is a SGEF protein corresponding to the protein encoded by the SGEF gene located at 3q25.2.

14. A method of diagnosis at least one disease state selected from among retinal macular anomaly (RMA), corpus callosum anomaly, hippocampus anomaly, liver disease, immune dysfunction, or feverless response to infection, comprising identifying a defect in an SGEF gene located at 3q25.2 or reduction in concentration level of an SGEF protein.

15. The method of claim 14, wherein said diagnosis of an individual comprises the detection of a defect in the SGEF gene located at 3q25.2 in a consanguineous other-individual or manifesting clinical or physical anomaly corresponding to at least one disease state from among retinal macular anomaly (RMA), corpus callosum anomaly (CCA) liver disease, immune dysfunction and feverless response to an infection.

16. A method of prevention or treatment of atherosclerosis or arteritis, comprising the systemic or local modulation of the SGEF levels or activity in a mammal.

17. A method of treatment or prevention of a medical condition, comprising administration of an agent to reduce SGEF presence or activity in a mammal, said agent being administered systemically or locally.

18. The method of claim 17, wherein said medical condition is a cancer or tumor growth.

19. The method of treatment or prevention of cancer or tumor growth of claim 17, wherein said cancer or tumor is a prostrate, brain, breast, ovary or liver cancer or tumor.

20. The method of claim 17, wherein said medical condition is inflammatory or auto-inflammatory or auto-immune diseases, illnesses or processes.

21. The method of claim 17, wherein said medical condition is increased intraocular pressure or glaucoma.

22. A method of preservation or preparation of an organ for transplantation, wherein said organ is exposed to a solution comprising SGEF protein or protein variant.

23. The method of claim 22, wherein said organ is liver.

24. A kit for treatment of a patient comprising at least a functional domain of an SGEF protein and a pharmaceutical excipient.

25. The kit of claim 24, wherein said protein is provided as a gene for expression in a mammal.

26. A kit for treatment of a patient comprising an inhibitor of at least an SGEF protein functional domain and a pharmaceutical excipient.


SEQUENCE LISTING (5 sequences)

SEQ ID NO: 1 - protein, 751 aa, Homo sapiens
MDGESEVDFSSNSITPLWRRRSIPQPHQVLGRSKPRPQSYQSPNGLLITDFPVEDGGTLLAAQIPAQVPTASDSRTVHRSPLLLGAQRRAVANGGTASPEYRAASPRLRRPKSPKLPKAVPGGSPKSPANGAVTLPAPPPPPVLRPPRTPNAPAPCTPEEDLTGLTASPVPSPTANGLAANNDSPGSGSQSGRKAKDPERGLFPGPQKSSSEQKLPLQRLPSQENELLENPSVVLSTNSPAALKVGKQQIIPKSLASEIKISKSNNQNVEPHKRLLKVRSMVEGLGGPLGHAGEESEVDNDVDSPGSLRRGLRSTSYRRAVVSGFDFDSPTSSKKKNRMSQPVLKVVMEDKEKFSSLGRIKKKMLKGQGTFDGEENAVLYQNYKEKALDIDSDEESEPKEQKSDEKIVIHHKPLRSTWSQLSAVKRKGLSQTVSQEERKRQEAIFEVISSEHSYLLSLEILIRMFKNSKELSDTMTKTERHHLFSNITDVCEASKKFFIELEARHQNNIFIDDISDIVEKHTASTFDPYVKYCTNEVYQQRTLQKLLATNPSFKEVLSRIESHEDCRNLPMISFLILPMQRVTRLPLLMDTICQKTPKDSPKYEVCKRALKEVSKLVRLCNEGARKMERTEMMYTINSQLEFKIKPFPLVSSSRWLVKRGELTAYVEDTVLFSRRTSKQQVYFFLFNDVLIITKKKSEESYNVNDYSLRDQLLVESCDNEELNSSPGKNSSTMLYSRQSSASQSPLYSDSP

SEQ ID NO: 2 - protein, 446 aa, Homo sapiens
MDGESEVDFSSNSITPLWRRRSIPQPHQVLGRSKPRPQSYQSPNGLLITDFPVEDGGTLLAAQIPAQVPTASDSRTVHRSPLLLGAQRRAVANGGTASPEYRAASPRLRRPKSPKLPKAVPGGSPKSPANGAVTLPAPPPPPVLRPPRTPNAPAPCTPEEDLTGLTASPVPSPTANGLAANNDSPGSGSQSGRKAKDPERGLFPGPQKSSSEQKLPLQRLPSQENELLENPSVVLSTNSPAALKVGKQQIIPKSLASEIKISKSNNQNVEPHKRLLKVRSMVEGLGGPLGHAGEESEVDNDVDSPGSLRRGLRSTSYRRAVVSGFDFDSPTSSKKKNRMSQPVLKVVMEDKEKFSSLGRIKKKMLKGQGTFDGEENAVLYQNYKEKALDIDSDEESEPKEQKSDEKIVIHHKPLRSTWSQLSAVKRKVILIVGFMEMKDGRLRGGK

SEQ ID NO: 3 - protein, 110 aa, Homo sapiens
MDGESEVDFSSNSITPLWRRRSIPQPHQVLGRSKPRPQSYQSPNGLLITDFPVEDGGTLLAAQIPAQVPTASDSRTVHRSPLLLGAQRRAVANGGTASPEYRAASPRLRR

SEQ ID NO: 4 - protein, 154 aa, Homo sapiens
MKSLILLQGRTAPQCSIQDRALPVSHLFTLTVLSNHANEKVEMLLGAETQSERARWITALGHSSGKPPADRTSLTQVEIVRSFTAKQPDELSLQVADVVLIYQRVSDGWYEGERLRDGERGWFPMECAKEITCQATIDKNVERMGRLLGLETNV

SEQ ID NO: 5 - protein, 137 aa, Homo sapiens [sequence omitted]